(19) United States
(12) Patent Application Publication
(10) Pub. No.: US 2015/0265191 A1
(43) Pub. Date: Sep. 24, 2015

(54) ELECTROCHEMICAL DETERMINATION OF INFECTION

(71) Applicants: Philip HARDING, Newport Beach, CA (US); Craig KESHISHIAN, Newport Beach, CA (US); INDICATOR SYSTEMS INTERNATIONAL, INC., Newport Beach, CA (US)

(72) Inventors: Philip Harding, Newport Beach, CA (US); Craig Keshishian, San Clemente, CA (US)

(21) Appl. No.: 14/443,348

(22) PCT Filed: Nov. 18, 2013
(86) PCT No.: PCT/US13/70631

§ 371 Date: May 15, 2015

Related U.S. Application Data
(60) Provisional application No. 61/727,615, filed on Nov. 16, 2012.

(51) Int. Cl. A61B 5/1477; A61B 5/145; A61B 5/00; A61B 5/053; A61F 13/00

(52) U.S. Cl. CPC A61B 5/1477; A61B 5/0537; A61F 13/00; A61B 5/7225; A61B 5/0004; A61B 5/14539; A61B 5/742; A61B 5/7282; A61B 5/7203; A61B 2560/0412; A61B 2562/0214; A61B 2560/0475; A61B 2560/0247

(57) ABSTRACT

Provided herein are devices including a wrapping, such as a bandage or a food wrapper, that can detect incipient infection at wounds or incipient contamination in food by voltage, resistance, and/or current. Methods for detecting incipient microbial infections and incipient microbial contaminations are also included herein.

### FIELD OF THE INVENTION

The present technology relates to detecting incipient microbial infection or contamination in specimen, for example, at wounds and in food. Specifically, this disclosure relates to systems and methods for measuring changing voltage, resistance, and/or current caused by increasing microbial loads.

### BACKGROUND

Active microbial growth and especially bacterial growth can lead to foodborne illness, wound infections, and other illnesses and diseases. Bacterial infections are a major concern in hospitals and other health care settings, particularly in an era of increasing prevalence of antibiotic-resistant bacteria. Proliferation of food-borne pathogens or microbes, particularly, bacteria, is also a major concern for restaurants, grocery stores, and other suppliers and servers of food. Yet means of monitoring active microbial growth remain limited. The presence of pathogenic microbes may be monitored visually, for example, by looking for signs of food spoilage such as discoloration or odor changes, or by looking for signs of infection in a patient such as inflammation, pus production, or fever. Visual monitoring is not possible in many setting though and detection is unreliable and only possible after the bacteria have significantly proliferated and begun to cause harm in the form of noticeable spoilage or infection. The presence of pathogenic bacteria may also be monitored through various cultures whereby a tissue or fluid sample is taken from a specimen, plated, incubated, and monitored for microbial growth in a laboratory setting. Such monitoring cannot be performed in real-time; in fact, obtaining test results often takes days. Thus, there is a need for reliable and real-time monitoring of changing microbial levels in various settings.

### SUMMARY

Embodiments disclosed herein generally relate to improved devices, systems, and methods for monitoring microbial proliferation at the location of a specimen and identifying incipient microbial infection or contamination. Of particular concern is incipient bacterial infection. However, the invention can also be used to monitor the growth of fungi, yeast and other microbes which respire.

Microbes such as bacteria present in meat, fish, fruits, vegetables, or the human body, consume glucose, fructose, or other sugars or carbon-based molecules, and release carbon dioxide (CO₂) as one of its byproduct. Thus, a patient with rising levels of microbes in the bloodstream will outgas CO₂ from any wound he or she has, such as a catheter wound. Similarly, a patient with microbes growing in or around a flesh wound will outgas CO₂ from that wound. Meat, fish, and other foods on which microbes are proliferating will also emit CO₂. The CO₂ chemically reacts with moisture, for example, on the skin's surface, to form a small amount of an acidic electrolyte, carbonic acid. This causes the acidity of the surface moisture to increase, thereby decreasing the pH of this surface moisture. It is thus possible to detect increasing levels of bacteria or other microbes by detecting corresponding decreases in pH levels. For example, by covering a wound with a pH-indicating material, such as a pH-indicating laminate disposed on a bandage, it is possible to identify that microbes have proliferated when the pH-indicating material changes color. This process is useful but requires either a transparent bandage or to have the clinician observe the color change on a bottom side of the bandage. Moreover, the utility is limited, because pH-indicating material are threshold indicators, only providing an indication as to whether a specific threshold value has been reached upon production of the required color. Still further, if the color produced is red to reddish-brown as many are, the clinician must distinguish between blood oozing from the wound and a color change due to the change in pH.

A need exists for devices, systems, and methods for monitoring changing microbial levels at the site of a specimen, preferably in vivo and in real-time. A need exists for devices, systems, and methods that can provide an indication of incipient microbial infection or contamination. A need exists for devices, systems, and methods that enable physicians, technicians, patients, restaurateurs, or food consumers to easily identify microbe proliferation, particularly, increases in bacteria levels over time and/or increased rates of change in bacteria levels. Various embodiments disclosed herein may fulfill one or more of these needs.

### SUMMARY OF THE INVENTION

Provided herein is a device for detection of incipient microbial infections. Specifically, provided herein is an electronic device for placement on a specimen of interest, such as a wound or food product, which detects changes in voltage, resistance, or current in a circuit and thereby recognizes corresponding changes in pH levels. The occurrence of microbial growth can then be ascertained from said changing pH levels by a person, a remote computing device, a wirelessly connected computing device, or an attached/on-board computing device.

One aspect of the current disclosure is directed to a device in the form of a wrapping, such as a dressing, a bandage, a film, a food wrapper, a sheet, or simply a wire. In various embodiments, a cathode and an anode are in electronic communication with the device. At least a portion of the device is in contact with fluid at risk of microbial growth, and the presence of microbial growth is readily measured by a change in the voltage between the cathode and the anode relative to the voltage present when the device is not in contact with the infected or contaminated fluid. In such situations, the cathode and anode in contact with the fluid being tested for active microbial growth is sometimes referred to herein as the “test cathode and anode”.

In certain embodiments, the device further includes a cathode and an anode to be positioned remote from the site of the fluid being tested for active microbial growth. This cathode and anode is sometimes referred to herein as the “reference cathode and anode” and acts as a control. The voltage from the reference cathode and anode serves as a control so that small changes in voltage between the test cathode and anode and the reference anode and cathode accurately measure a change in pH and, accordingly, a strong characteristic of an incipient active microbial infection.

In certain embodiments, the device further includes an alarm that alerts an individual, such as for example, a patient or caregiver, that a patient's wound is under an incipient active infection. In other embodiments, the device further includes an alarm that alerts an individual that a food product is experiencing active microbial growth.

Also provided herein are methods for detecting infection at a wound or contamination in food by using the devices of this invention.

Another aspect of the current disclosure is directed to a device for detecting an incipient bacterial infection or contamination. In various embodiments, the device includes a pair of bandages, each bandage having a sheet of metal or conducting polymer on a bottom side. Each bandage in the pair forms a single half-cell and both bandages include the same metal or conducting polymer. In various embodiments, the bandages are configured for placement on a patient, such that when a first of the pair is placed directly over a wound and a second of the pair is placed on nearby skin over a wound-free area, the skin acts as a low impedance conductor and a connected circuit can detect a voltage across the half-cells. In some embodiments, the device is configured to monitor the detected voltage over time and output an alert when a change in voltage, such as an increase in voltage, occurs. In various embodiments, an increase in voltage is indicative of an increase in the bacterial load. It is possible, of course, to employ a single bandage containing two half-cells where one of the half-cells is placed in the adhesive portion of the bandage sufficiently removed from the wound site such that it functions independent of that other half-cell placed over the wound site.

The device of some embodiments, such as any of the embodiments described above or elsewhere herein, also include a detection circuit. The detection circuit of some embodiments includes, at least, an amplifier, a processor, and memory. In some embodiments, the detection circuit is separably connected to the wrapping via a connector. In other embodiments, the detection circuit is permanently disposed on or in the wrapping. In various embodiments, the detection circuit additionally includes a wireless transmitter.

An additional aspect of the disclosure is directed to a system, which includes the detection device described in this section or elsewhere herein and a receiving circuit. In some embodiments, the receiving circuit includes a wireless receiver, a processor, a memory, and an output display. In various embodiments, the receiving circuit is configured to further process and/or analyze voltage signals recorded by the wrapping.

### BRIEF DESCRIPTION OF THE DRAWINGS

FIG. 1 depicts a schematic bottom view of one embodiment of a device for monitoring changes in pH at the site of an open wound.

FIG. 2 depicts a schematic bottom view of another embodiment of a device for monitoring changes in pH at the site of an open wound.

FIG. 3 depicts a schematic bottom view of an additional embodiment of a device for monitoring changes in pH at the site of an open wound.

FIG. 4 depicts a schematic bottom view of one embodiment of a device for monitoring changes in pH on food surfaces.

FIG. 5 depicts a block diagram of a circuit for detecting changes in pH from detected changes in voltage, resistance, or current.

FIG. 6 depicts another block diagram of a circuit for detecting changes in pH from detected changes in voltage, resistance, or current.

FIG. 7 depicts a schematic bottom view of one embodiment of a device for monitoring changes in pH at the site of an open wound.

FIG. 8 depicts a schematic bottom view of another embodiment of a device for monitoring changes in pH at the site of an open wound.

FIG. 9 depicts a schematic bottom view of an additional embodiment of a device for monitoring changes in pH at the site of an open wound.

FIG. 10 depicts a schematic front view of the device embodiment of FIG. 9.

FIG. 11 depicts a block diagram of a circuit for detecting changes in pH from detected changes in voltage, resistance, or current.

FIG. 12 depicts a line graph of typical pH values observed at an acute wound over time.

FIG. 13 depicts a flow chart of one method of determining the presence of active microbial growth in or on a specimen.

### DETAILED DESCRIPTION

In the following detailed description, reference is made to the accompanying drawings and the accompanying appendix, which form part of the present disclosure. The embodiments described in the drawings and description are intended to be exemplary and not limiting. As used herein, the term “exemplary” means “serving as an example or illustration” and should not necessarily be construed as preferred or advantageous over other embodiments. Other embodiments may be utilized and modifications may be made without departing from the spirit or the scope of the subject matter presented herein. Aspects of the disclosure, as described and illustrated herein, can be arranged, combined, and designed in a variety of different configurations, all of which are explicitly contemplated and form part of this disclosure.

Unless otherwise defined, each technical or scientific term used herein has the same meaning as commonly understood by one of ordinary skill in the art to which this disclosure belongs. In accordance with the claims that follow and the disclosure provided herein, the following terms are defined with the following meanings, unless explicitly stated otherwise.

The term “about” or “approximately,” when used before a numerical designation or range (e.g., pressure or dimensions), indicates approximations which may vary by (+) or (−) 5%, 1% or 0.1%.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a device or method consisting essentially of the elements as defined herein would not exclude other materials or steps that do not materially affect the basic and novel characteristic(s) of the claimed invention. “Consisting of” shall mean that the devices, systems, and methods include the recited elements and exclude anything more than a trivial or inconsequential element or step. Embodiments defined by each of these transitional terms are within the scope of this disclosure.

As used herein in the specification and claims, the singular form “a,” “an” or “the” include both singular and plural references unless the context clearly dictates otherwise. For example, and without limitation, “a cathode” includes one or more cathodes. At times, the claims and disclosure may include terms such as “a plurality,” “one or more,” or “at least one;” however, the absence of such terms is not intended to mean, and should not be interpreted to mean, that a plurality is not conceived.

As used herein, “specimen” shall refer to any organism or any portion or byproduct thereof at risk of microbial infection. For example, a specimen may refer to an open wound on a human or animal patient, a piece of fruit, a vegetable, a package of fruits or vegetables, eggs, or a cut of meat, fish, or poultry, or milk, cheese, or other milk products.

As used herein, “wound” shall refer to an open or closed wound, such as, for example, a scrap, laceration, gunshot wound, open sore, or other open flesh wound, a wound resulting from surgery such as surgical incision sites or catheter sites, and/or a wound closed by sutures, staples, or other closure means.

As used herein, “a wrapping” shall refer to any bandage, food wrapper, film, dressing, sheet, substrate, or other thin material or layer configured to be adhered to, or wrapped around or partially around, a specimen, or any wire or probe configured to be inserted at least partially within a specimen.

As used herein, “bottom side” shall refer to the face of the wrapping, which in use, is closest to the specimen. In various embodiments, the bottom side is the face which touches the surface of the specimen.

As used herein, “top side” shall refer to the face of the wrapping, which in use, is furthest and/or facing away from a surface of the specimen. In various embodiments, the top side is the face which is facing, and exposed to, the ambient environment.

“Infected or contaminated fluid” or just “infected fluid,” as used herein, refers preferably to an aqueous fluid in which a microbe is actively growing. As also used herein, “infected” and “infection” each refers to microbial loads in a specimen, which are above and beyond a safe threshold level.

The term “microbe” refers to any microbe which respires during growth so as to produce carbon dioxide and, hence, a change in pH. Microbes include bacteria, fungi, yeast and the like. However, in a preferred embodiment, the microbes of interest are bacterial in nature. For the purpose of this application, reference is sometimes made to “bacteria” or bacterial” and these are illustrative in nature and not meant to exclude fungus and yeast.

Changes in pH values on the surface of a specimen may be indicative of an incipient active bacterial infection and may be detected through a variety of approaches using various devices, systems, and methods described herein.

### pH Meter Approach

One approach disclosed herein involves the miniaturization of each of the two electrodes found in a pH meter. In various embodiments, the two electrodes are laminated to, or otherwise disposed on, the bottom side of a wrapping. In some embodiments, electrodes are printed or plated onto the wrapping using known microprinting, print, and/or plating techniques.

Once affixed to the wrapping, one electrode is located in the region of the specimen and positioned such that if bacteria are present, and acidity is thereby created, the electrode is located in or on the region of the acidic electrolyte. The other electrode is located nearby so as to complete the electrical connection while remaining isolated from the bacteria-induced acidity. The actual detection electronics can be placed external to the wrapping by means of a connector. Alternatively, some or all of the detection electronics may be integrated onto the wrapping.

### Voltaic Cell Approach

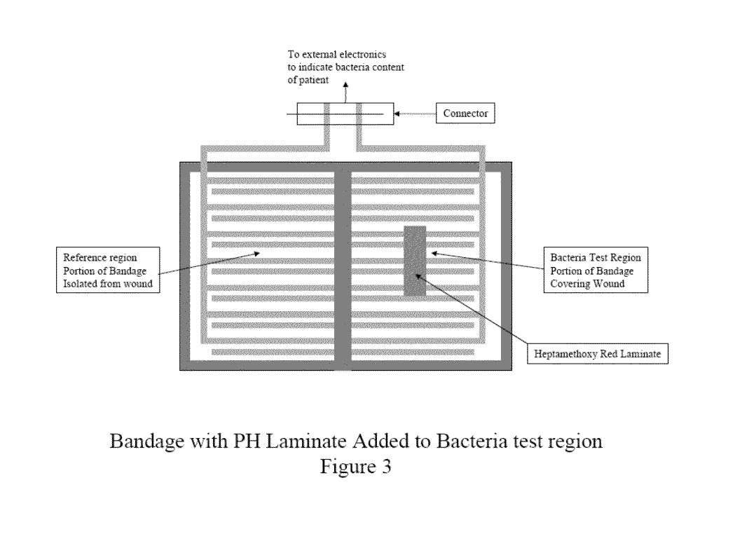

One approach disclosed herein employs a voltametric approach of measuring surface moisture acidity. For example, and without limitation, FIG. 3 depicts the bottom side of a wound bandage containing an electrical connection and pH detecting laminate material.

In the embodiment of FIG. 3, the wrapping contains two regions, the Bacteria Test Region and the Reference Region, each of the regions separated by a barrier, such as a gauze, foam, and/or adhesive barrier, to prevent the fluid being tested for active microbial growth in the Bacteria Test Region from migrating to the Reference Region. The Bacteria Test Region is in the proximity of the wound and will develop a higher-acidity electrolyte than the Reference Region. The Reference Region is adjacent to, but kept separate from, the Bacteria Test Region. In some embodiments, there is a surrounding adhesive, which securely but non-permanently couples the bandage on to the wound of the patient.

The Bacteria Test Region can optionally contain a pH sensing laminate, which will change color when the pH of the specimen reaches or crosses a threshold pH level. In the depicted example, the pH sensing laminate is shown as Heptamethoxy Red Laminate, which will turn red if the electrolyte in the Bacteria Test Region is sufficiently acidic. In other embodiments, any other pH sensing material having a desirable color-changing threshold, such as, for example, a layer of Hexamethoxy Red, may be used. In various embodiments, the pH sensing laminate is positioned on a bottom side of the wrapping, disposed above or below a portion of the detection electrodes. In some embodiments, the wrapping is formed of a transparent material such that the pH sensing laminate is viewable from a top side of the wrapping.

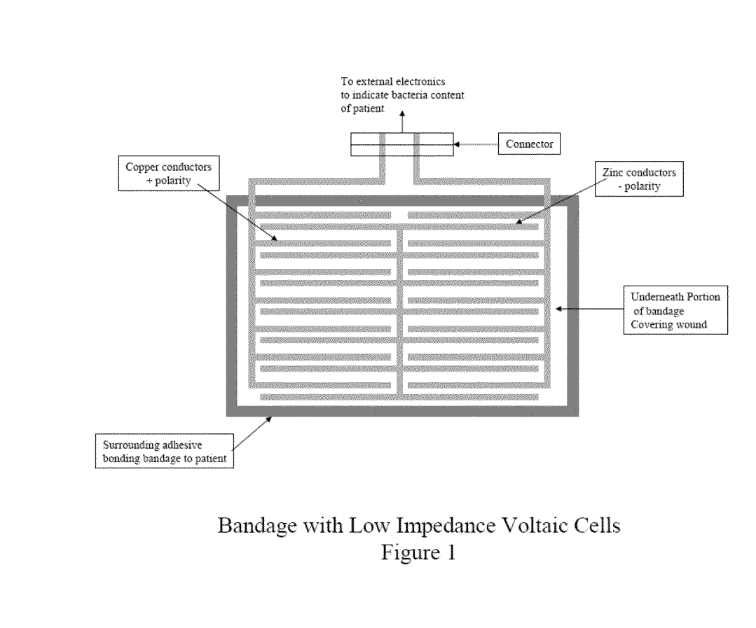

In various embodiments, the electrodes of the Bacteria Test Region and the Reference Region are constructed as shown in FIG. 1, such that a copper cathode and a zinc anode are present to form a low impedance voltaic cell. In the depicted embodiment, a plurality of copper wires, rods, strips etc. (“copper electrodes”) are connected together and a plurality of zince wires, rods, strips, etc. (“zinc electrodes”) are connected together. The electrodes are spaced such that the copper and zinc electrodes are in an alternating arrangement on a bottom side of the bandage. In various embodiments, the electrode pattern of the Reference Region is the same, substantially the same, a mirror image, or substantially a mirror image of the electrode pattern of the Bacteria Test Region. The arrangement results in all the copper electrodes being connected in parallel and all the zinc electrodes being connected in parallel.

Without being bound by theory, two nodes of dissimilar metals in the presence of a current-carrying electrolyte develop a cell voltage; the voltage being a function of the metals, their shape, how far apart the dissimilar metals are, and the conduction capability of the electrolyte. In the example of FIG. 1, the copper node will be the positive (+) terminal and the zinc node will be the negative (−) terminal. However, there are many combinations of dissimilar metals, metal alloys, and conducting polymers that can be used. The arrangement shown in FIG. 1 actually develops many cells, all are interconnected in a parallel configuration, resulting in a group cell with a lower impedance. The wrapping can be constructed with as many copper and zinc lines as is possible with the limit being that sufficient distance between lines must be maintained to prevent erroneous connection between adjacent copper and zinc lines.

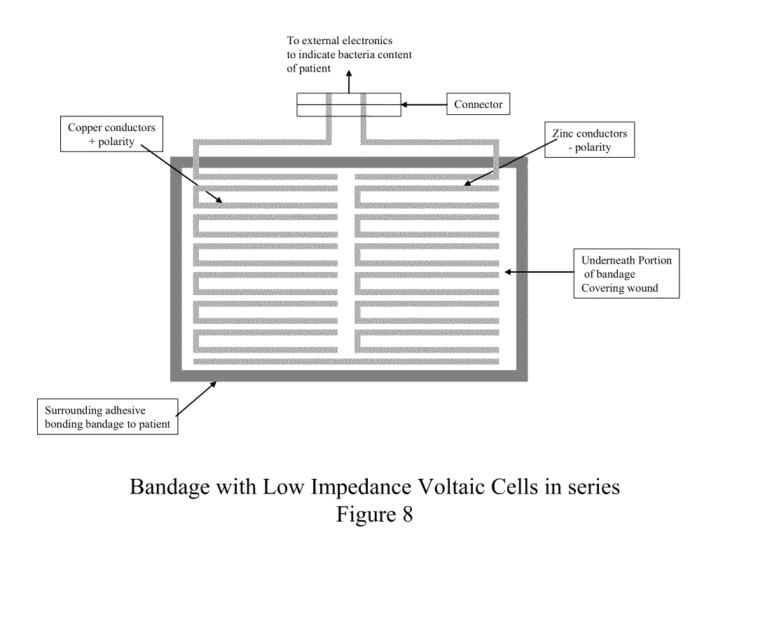

In another embodiment, a plurality of voltaic cells formed of dissimilar metals, metal alloys, or conducting polymers may be connected in series. A bandage with low impedance voltaic cells in series is schematically illustrated in FIG. 8. In such an embodiment, a plurality of copper electrodes and zinc electrodes are disposed in a spaced and alternating arrangement on a bottom side of a wrapping. A copper electrode is physically connected to a zinc electrode to form an electrode pair. A plurality of electrode pairs are provided on the wrapping. In various embodiments, the electrode pattern of the Reference Region is identical to or a mirror image of the electrode pattern of the Bacteria Test Region.

It is contemplated that the voltage developed will increase as the acidity content of the fluid being tested for active microbial growth increases. Such an increase in voltage corresponds to an increase in bacteria since the acidity of the fluid increases with increased bacterial out-gassing of CO₂, which increases with increasing bacterial loads. Thus, such an increase in voltage can act as an indicator that there is an incipient bacterial infection or contamination.

Because the voltage can vary with temperature, sweat, muscle movement, and many other variables, in various embodiments, the identical arrangement of two nodes, such as zinc and copper nodes, is provided in the Reference Region as mentioned above. Such nodes are positioned in a region where the skin's surface moisture will be free of the bacteria outgas but where other physiological conditions such as sweat content and skin temperature will be substantially identical to conditions at the Bacteria Test Region. In some embodiments, the cells in the Reference Region are directly connected to the cells in the Bacteria Test Region but are of opposite polarity; thus, any voltage developed in the Reference Region is subtracted from the voltage in the Bacteria Test Region. This subtraction eliminates or largely eliminates the effect of the variables since any artifacts (i.e., any voltage signal resulting from these unwanted variables) will be present at both the Bacteria Test Region and the Reference Region.

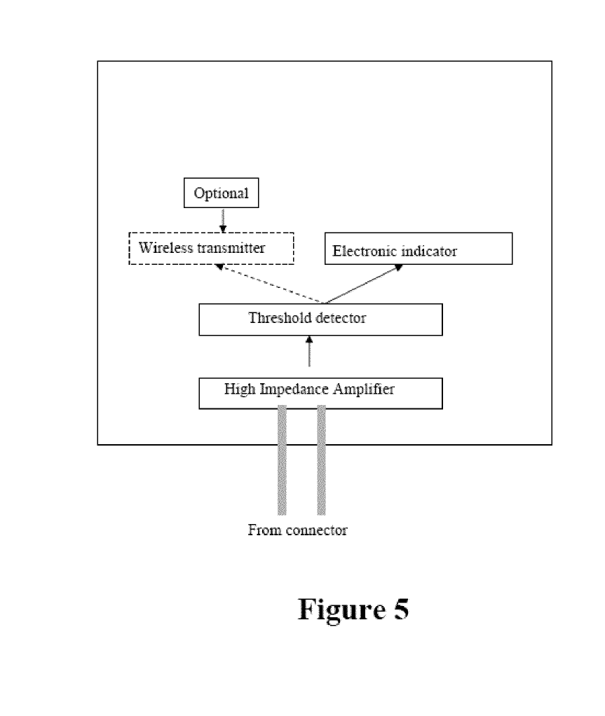

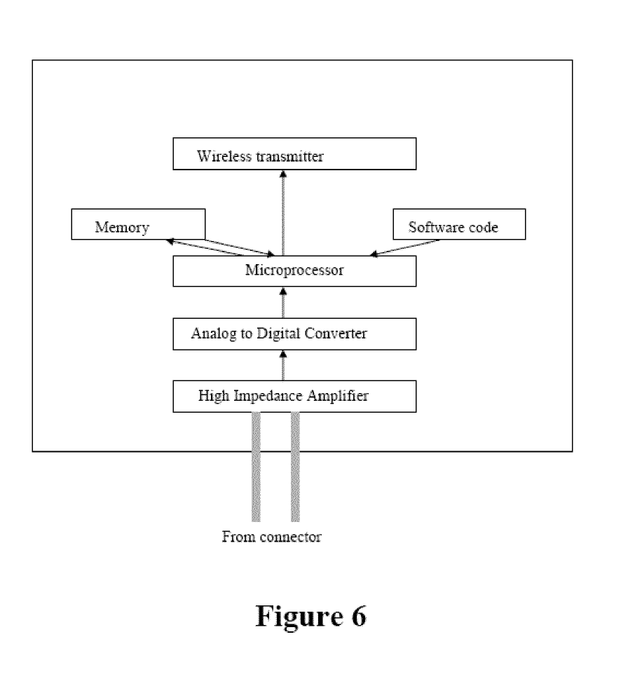

For example, if the wound is free of the out-gassing bacteria, the net voltage seen at the connector is 0 at any temperature and any skin moisture content. A voltage is only developed if carbon dioxide out-gassing is present to increase the acidity of the moisture under or near the Bacteria Test Region. This change in voltage is contemplated to be detected with appropriate electronics, such as, for example, those shown in FIGS. 5 and 6.

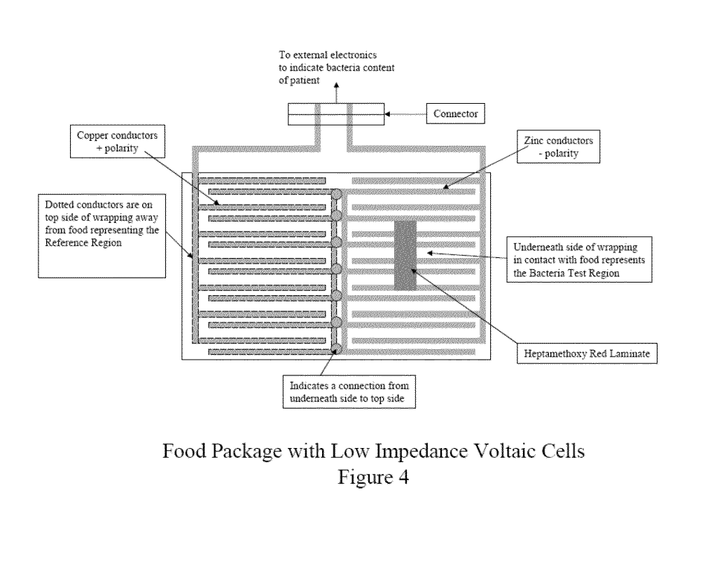

FIG. 4 schematically provides a system to test bacteria contamination in a food package. Such a system may have some or all of the features of the wound bandages described elsewhere herein.

In some embodiments of a food package, it is necessary to separate the Reference Region entirely from the bottom side since the bacterial contamination may not be limited to a specific portion of the specimen. That is, the risk of bacterial contamination is widespread; bacteria may proliferate in a plurality of locations of the food surface. Thus in some embodiments, the Reference Region is relocated to the top side of the wrapping. The wrapping of various embodiments is a thin film or membrane, thus relocating the Reference Region to the top side of the wrapping has a negligible difference in temperature and potentially a negligible difference on the values of other variables. In some embodiments, a plurality of Bacteria Test Region and Reference Region pairs are positioned on a food wrapping so that bacteria proliferation can be monitored at a plurality of sites, such as, for example, along a length of a food product. In another embodiment, the food wrapping is a box or other food container having a plurality of test electrodes positioned therein, in the form of wires or rods. Such electrodes may extend into a food product, such as ground beef, at a plurality of locations so as to allow monitoring throughout all or substantially all of the food product. Such an embodiment may enable a user to identify incipient microbial contamination, such as incipient E. coli contamination, regardless of where in the food product the developing contamination is originating.

FIG. 5 depicts an electronic detection circuit, configured to enable the detection of increased acidity. Such a circuit may be used with the bandage of FIG. 4 or other wrapping embodiments described herein. In such an embodiment, the circuit is configured to detect changes in pH levels and can detect when a bacterial load has crossed a threshold level, such as an unsafe threshold level or any earlier threshold level indicating that an incipient infection or contamination is forming. In some embodiments, an electronic indicator outputs a signal when a threshold has been crossed. In some such embodiments, the electronic indicator is a light, such as an LED diode. In other embodiments, the electronic indicator may be an audible alarm or a display screen capable of presenting a warning message or image.

FIG. 6 depicts a detection circuit employing an Analog to Digital converter and microprocessor so that a large variety of data can be obtained related to:
(a) time of occurrence of increased bacteria/decreased pH
(b) date of occurrence
(c) rate of change of bacteria content, which enables a determination prior to a crisis
(d) location of occurrence
(e) levels of bacteria, and/or
(f) comparison between periods of time. For example, by comparing the reading when the bandage is first applied to later time periods, the effects of most variables will be eliminated.

FIG. 6 is one embodiment of a detection circuit. In addition to the ADC and the microprocessor, a high impedance amplifier and wireless transmitter are present. Memory is also present, either in the microprocessor or separate. The memory stores software code that the microprocessor can read and execute, and optionally, also receives and stores data that the microprocessor writes into the memory. In other embodiments, one or other electrical components may be substituted or supplemented. For example, in some embodiments, the circuit also includes a pre-amplifier, a low-pass filter, a high-pass filter, and/or a band-pass filter.

The detection circuit shown in FIGS. 5 and 6 can be separate from the wrapping and reversibly attachable by application of a connector (such as cables and a connection interface), as shown in the illustrations, or it can be designed as part of the wrapping, eliminating the need of an external connector. This choice depends upon the application. For example, if the application is for a bulk-contained quantity of food, it may be better to have the adjunct electronics as part of the wrapping.

### Thermocouple Approach

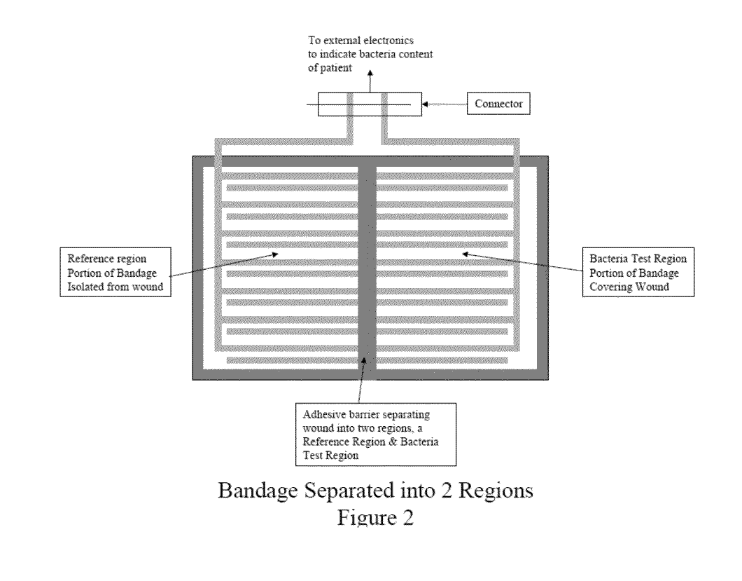

The circuitry illustrated in FIGS. 1 through 6 can be further augmented with the addition of thermocouples, also printed or plated onto the bandage or food wrapping. Such can add the capability of electronically measuring the change in temperature relative to some reference location. Without being bound by theory, it is known that different metals or metal alloys will generate a different voltage along their lengths if there is a temperature differential along this same length. The amount and polarity of the voltage depends on the temperature differential as well as the metal chosen. However, the voltage levels will be less than millivolts as compared to the Voltaic Approach which can be much more robust.

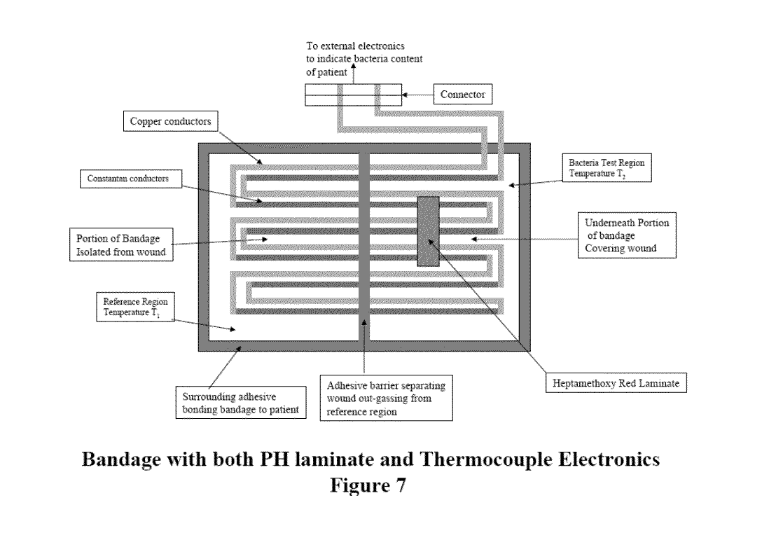

FIG. 7 illustrates usage of this concept. A copper conductor is connected to a constantan conductor in the Bacteria Test Region, as shown in the lower right corner. The two metals remain separated until the Reference Region. In the Reference Region the constantan is connected to another copper conductor and the copper conductor is connected to another constantan conductor; both conductors are brought back to the Bacteria Test Region. This type of interchange of conductor pairs continues back and forth between regions until the conductors are brought out to a connector. By means of this type of connection, a measurable voltage will be developed across the two conductors at the connector if there is a temperature differential between T₁ and T₂. The metals do not have to be the ones chosen in the illustration. In fact, they can be the same as the metals used for the Voltaic portion of the circuits; the major criteria is that the chosen metals behave differently with temperature change. The sensitivity of the thermocouple will also be increased by increasing the number of conductor pairs as much as is possible with the limit being that sufficient distance must be maintained between conductors to prevent erroneous connection between adjacent circuits.

This concept can be used for food packaging as well; however, in some such embodiments, the Reference Region will be on the top side of the wrapping instead of being separated by a barrier as in the bandage example described above.

The detection circuitry for the thermocouple can be very similar to the circuits shown in FIGS. 5 and 6. In fact, in some embodiments, some of the electronics are shared, for example, the microprocessor and wireless transmitter.

### Half Cell Approach

In some embodiments, an alternate approach is utilized in which two bandages are used, each bandage forming a single half-cell, and each half-cell containing the same metal, metal alloy, or conducting polymer. Although this example refers to two bandages each forming a single half-cell, in other embodiments, the device is formed of a single bandage having two portion, each portion forming a single half-cell. The approach compares the half-cell voltages developed when two bandages (or portions) of identical material and conducting nodes are placed on a patient—one directly over the wound area and the second placed nearby on the skin in a wound-free area.

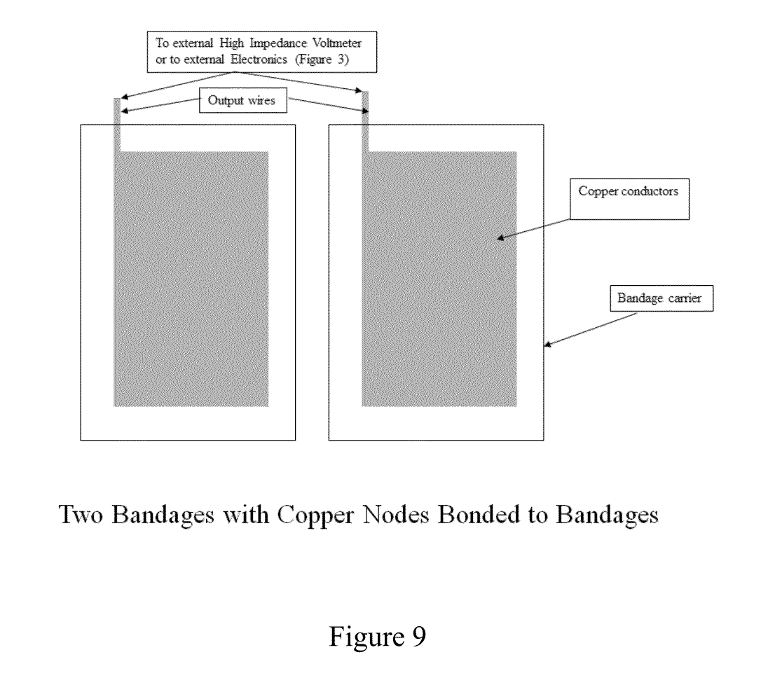

FIG. 9 depicts the bottom side of two such bandages containing the electrical connections. Each bandage is identical with a portion of each bandage containing a thin sheet of metal such as copper, zinc, silver, or any metal or conducting polymer that is not harmful to the patient but chemically reacts to the electrolytes over the skin of a patient.

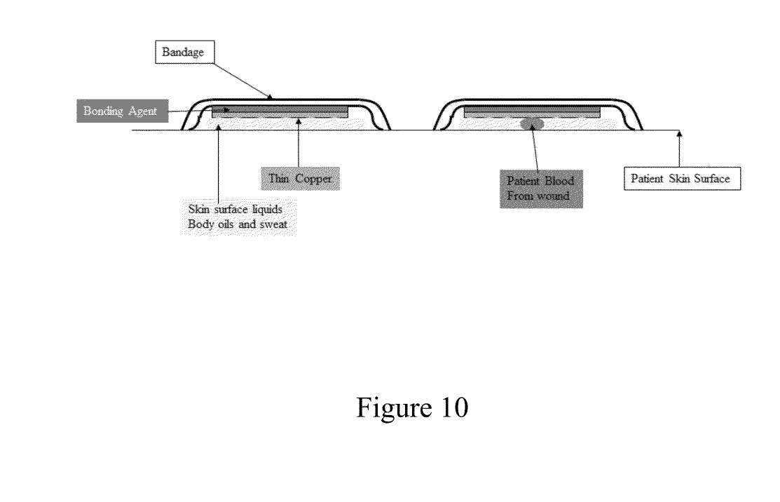

FIG. 10 shows a side view of the two bandages indicating how one is placed over the wound area of a patient and the other placed nearby in an area free of the wound. In use, the metal node in the bandage over the wound would be in direct contact with the blood of the wound, and the other nearby bandage metal node would be in contact with the patient's skin and surface fluids such as sweat.

Not to be limited by theory but using the well-known electrochemical theory, each of the two metal nodes will develop a voltage due to the liquids beneath the nodes. The difference between these two voltage will appear on the output wires because the body's skin between the bandages is conductive, behaving as a low impedance connection and thereby allowing the output wires to measure, primarily, the difference between the voltages developed by the metallic nodes. If the wound is relatively free of bacteria, the blood in the wound area will have a pH that is very similar to the pH in the wound-free area, such as, for example, a pH of approximately 7.0. Thus, the output voltage between the two output wires will be relatively low. Should the wound area develop a high content of bacteria, the pH level will drop well below 7, because the blood in the wound area will become acidic, raising the voltage of this cell. Then the difference in voltage between the two output wires will change, thereby indicating that the wound area is becoming infected.

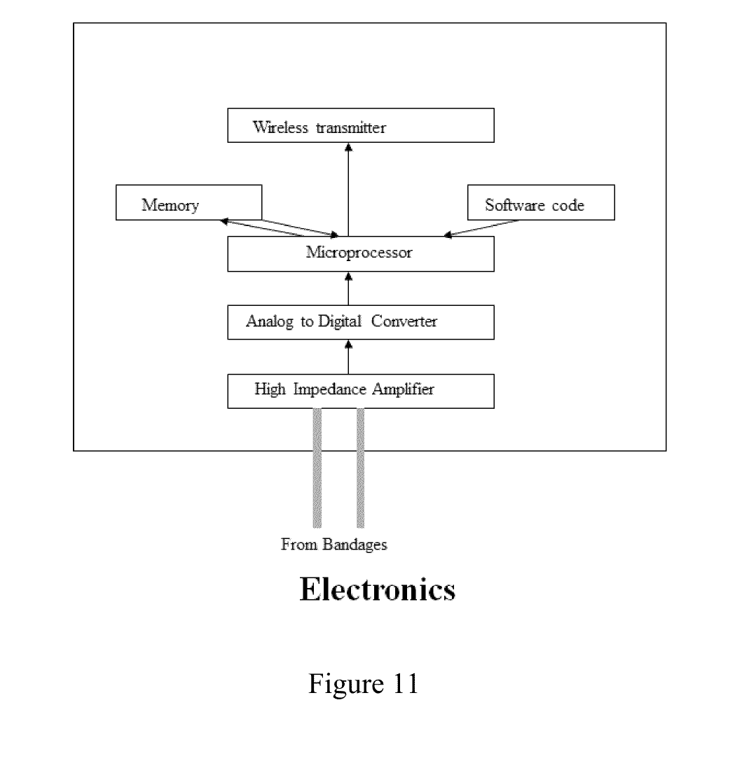

Because voltage can vary significantly with temperature, residual moisture, and many other variables, in some embodiments, such as the one shown in FIGS. 9 and 10, an identical arrangement of two bandages is provided, one over the wound and one in a wound-free Reference Region as mentioned above. The bandage (or bandage portion) positioned in the Reference Region acts as a control, and the difference in voltage between the active bandage over the wound and the control is recorded. With this arrangement, the voltage on the output wires will be independent or substantially independent of body temperature, ambient temperature, normal skin secretions, etc. since any artifact (i.e., any signal resulting from undesirable variables) will be present at both bandages, and thus, will be cancelled out when the voltage difference between the two bandages in measured. The remaining desired voltage can then be detected with appropriate electronics, such as those shown in FIG. 11.

FIG. 11 depicts an example detection circuit configured to enable the detection of an increased acidity in the wound area. This circuitry employs an analog-to-digital converter (ADC) and microprocessor so that a large variety of data can be obtained, such as for example:
(a) time of occurrence of increased bacteria in the wound area noticed by a change in voltage;
(b) date of occurrence of the voltage change;
(c) rate of change of bacteria content which enables a determination prior to a crisis;
(d) location of occurrence;
(e) levels of bacteria; and
(f) comparison between periods of time. By comparing the reading when the bandage is first applied to later time periods, the effects of most variables will be eliminated.

The detection circuit shown in FIG. 11 can either be separate by application of a connector between the electrodes on the bandage and other circuit components or, in some applications, the circuits can be included within the bandages.

In various embodiments described herein, it may be advantageous to include the detection circuit on a small chip imbedded on or in the wrapping or other adhesive patch. For example, in some embodiments, the detection circuit is connected to the electrodes by wires, and with the exception of said wires and electrodes, some or all of the electronics are housed on the top surface of the wrapping, for example, within a protective substrate or under a protective film or other casing.

In some such embodiments, the detection circuit includes, at least, a high impedance amplifier, an ADC, a microprocessor, memory, a power supply, and a display. The amplifier and ADC are provided to condition and process the signal. Additionally conditioning components such as filters may be provided. A system bus may also be provided which couples the various components and enables data and signals to be exchanged between the components. The system bus may operate on any of a number of known protocols. The memory stores a set of instructions, in the form of software code, which the microprocessor is configured to execute. Additionally, the memory may be configured to store data received from the electrodes, and optionally store date and time information received from a digital clock internal to the system, such that a time log of measurement data may be recorded and stored. In various embodiments, the power supply is a battery such as a rechargeable or replaceable battery. The display may include a digital display configured to display a numeric value, alphanumeric messages, and/or an image, or a light display configured to display various colored lights.

In some embodiments, the display is replaced with a wireless transmitter. In some such embodiments, the wireless transmitter includes a radiofrequency transmitter configured for Bluetooth, Wi-Fi, or near field communications.

In some embodiments, a system is provided, which includes the detection device (i.e., the wrapping with a detection circuit) described herein and a separately located receiver, such as a radiofrequency receiver. The receiver of various embodiments is part of a receiving circuit that also includes, at least, a processing unit and memory. The receiving circuit may also include a system bus which couples the various components of the receiving circuit and enables data and signals to be exchanged between the components. The system bus may operate on any of a number of known protocols. In some embodiments, the memory includes instructions in the form of software code, which can be executed by the processor in the receiving circuit to further process data received from the detection circuit's wireless transmitter. In some embodiments, the receiving circuit also includes a display, such as a touchscreen display, on which information about changes in pH and/or changes in the bacterial load may be presented to a user. The touchscreen display of various embodiments acts as both a user input device and a user output device. The user may be able to interact with the touchscreen to create, manipulate, annotate, and/or view a log of bacterial load. The user may be able to perform various functions with the aid of the receiving circuit and touchscreen, such as but not limited to: viewing changes in pH over a chosen time period, viewing rates of change in pH over a chosen time period, viewing results in a graphical or user-friendly format, setting personalized thresholds and alerts, etc. The receiving circuit is housed within a receiving device. In some embodiments, the receiving device is a smartphone, a tablet, or other mobile computing device. In other embodiments, the receiving device is a handheld accessory configured specifically to wirelessly couple to, and interrogate, the detection device.

As described above, in various embodiments of the detection circuit and receiving circuit, memory is provided. The memory may include random access memory (RAM), read only memory (ROM), or preferably, both. ROM may store a basic input/output system (BIOS) or other basic operating information system, while RAM generally stores the operating system (OS), application software, and data. Alternatively or additionally, the memory may include flash memory, electrically programmable ROM (EPROM), and/or electrically erasable programmable ROM (EEPROM).

As also described above, in various embodiments of the detection circuit and receiving circuit, a processor, such as a microprocessor, is provided. The processor of various embodiments includes a disk drive, for example, a hard disk drive. The hard disk drive is connected to the bus via a hard disk drive interface. A hard disk storing application modules and data may be installed on the disk drive. While a microprocessor is provided in exemplary embodiments described herein, in other embodiments, the detection functions may be implemented or performed with a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may also be implemented as a combination of computing devices, e.g., a combination of a DSP and a microprocessor, a plurality of microprocessors, one or more microprocessors in conjunction with a DSP core, or any other such configuration.

In one or more example embodiments, the functions described may be implemented in hardware, software, or firmware, or any combination thereof. Moreover, one of skill in the art will appreciate that various components disclosed as discrete components may together form a component; alternatively, any component disclosed as a single component may be formed of multiple components. For example: the memory may be coupled to the processor such that the processor can read information from, and write information to, the memory; alternatively, the memory may be integral to the processor. An ASIC may comprise both the processor and the memory. In some embodiments, a portion of the memory is integral to a microprocessor and a portion of the memory is a separate but coupled component.

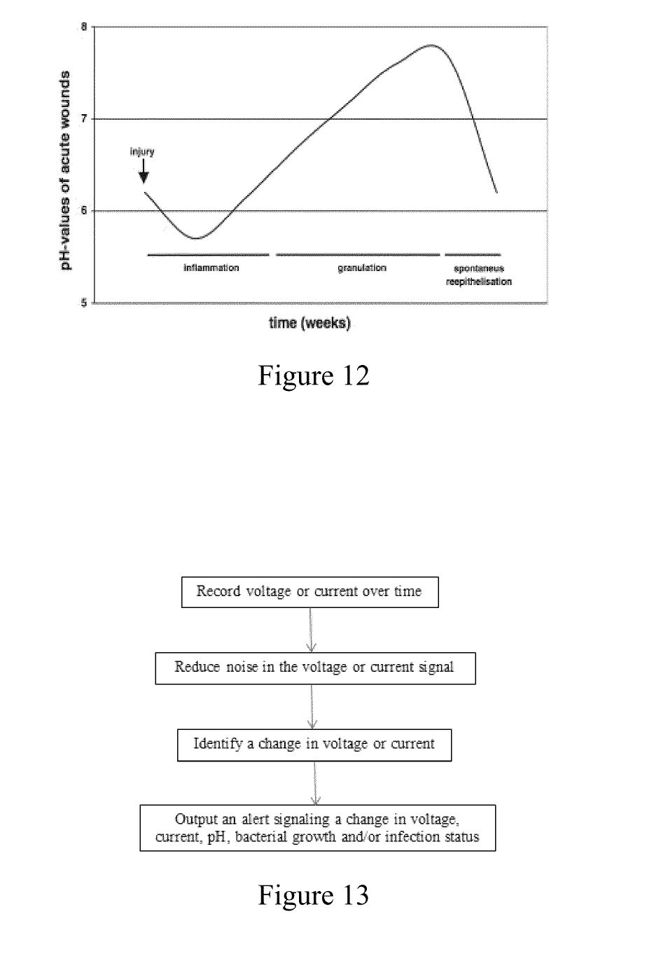

Advantageously, the circuitry in the various embodiments described herein is configured to detect drops in pH, which allows individuals to be alerted at the first signs of microbial infection in patients or the first sign of microbial contamination in food. As shown in the graph of FIG. 12, pH begins to drop before inflammation is evident. In fact, pH begins to drop at the earliest stages of infection/contamination. Thus, by monitoring for pH change, detection of incipient infections and contamination is possible. Various embodiments described herein allow for earlier detection than is possible with existing devices. In devices having just a pH-indicating laminate or other pH-indicating material that changes color at a threshold value, the threshold value is generally set such that infection is not detected until it is well established. Earlier detection allows for earlier and more successful treatment in patients. In the food industry, earlier detection significantly reduces the risk of a food-borne illness outbreak.

Various embodiments of a detection device, such as any of the embodiments disclosed herein may be used to identify active bacterial growth in or on a specimen and identify bacterial infections and contaminations in their initial developing stages. As shown in FIG. 13, in some embodiments, a method of identifying active microbial growth includes recording voltage at a specimen over time and identifying a change in voltage, where an increase in voltage is indicative of: a decrease in pH, an increase in active microbial growth, and an incipient microbial infection or incipient microbial contamination. In some embodiments the specimen is a wound in a patient; in other embodiments, the specimen is a food product. In some embodiments, voltage is recorded over a length of time and only changes in said voltage are analyzed and used as indicators of incipient microbial infection. Such a methodology may help eliminate artifacts in the signal caused by other variables since such variables may stay substantially constant over time.

In some embodiments, the method further includes placing a wrapping on the specimen, where the wrapping includes an active region and a reference region, each region having a positive node and a negative node. In some such embodiments, placing the wrapping on the specimen includes positioning the active region on the specimen and the reference region at a location near the specimen. For example, in some embodiments, placing the wrapping on the specimen includes placing the active region on a patient's skin so that it covers a wound and placing the reference region on the patient's skin at a location that is separate but adjacent to, or otherwise near, the wound. In some embodiments, a structure in the wrapping prevents fluid in the active region from flowing or seeping into the reference region. The nodes of the active region and reference region are connected to a detection circuit, if not already connected. In some embodiments, the method further includes subtracting a reference voltage signal recorded at the reference site from an active voltage signal recorded at the active site. This subtraction minimizes the effect of environmental variables on the active voltage signal and thereby reduces noise, improving the signal-to-noise ratio.

In other embodiments, the method further includes placing an active bandage or active bandage portion on a wound and placing an identical reference bandage or reference bandage portion on a skin surface separate from, but near, the wound. Each bandage or bandage portion includes a sheet of conducting material such as copper or other conducting metal, metal alloy or conducting polymer. The skin and surface fluids under the bandages or bandage portions act as a low impedance conductor. Both bandages or bandage portions are connected to the same detection circuit. In such embodiments, recording voltage involves recording the voltage difference between the active and reference bandages or bandage portions. By recording the difference, the effect of environmental variables on the resulting voltage signal is minimized.

In some embodiments, the detection circuit includes an ADC and the method additionally includes converting an analog voltage signal to a digital signal. The digital signal is sent to a computer for signal processing and analysis. In some embodiments, sending the digital signal to a computer comprises transmitting the digital signal to a remote computer via a wireless transmitter. In some embodiments, during processing, if an increase in voltage is detected, an output, such as an alarm, is generated to signal a decrease in pH, an increase in active microbial growth, and/or an incipient microbial infection or contamination. The alarm may be audible and/or visible.

In some embodiments of the method described above, current may be detected and used as an indicator of pH and microbial proliferation instead of voltage. As with voltage, an increase in current may indicate a decrease in pH, an increase in acidity, an increase in bacterial growth, and an incipient microbial infection or contamination.

Although the foregoing has included detailed descriptions of some embodiments by way of illustration and example, it will be readily apparent to those of ordinary skill in the art in light of the teachings of these embodiments that numerous changes and modifications may be made without departing from the spirit or scope of the appended claims.

1. A device comprising a wrapping, a cathode, and an anode, wired such that when the device is in contact with a fluid infected or contaminated with bacteria, the voltage between the cathode and the anode changes relative to the voltage present when the device is not in contact with the infected or contaminated fluid.

2. The device of claim 1, further comprising a reference cathode and a reference anode, wherein the voltage difference between the reference anode and cathode is not substantially changed with changing characteristics of the infected or contaminated fluid.

3. A device for monitoring an incipient bacterial infection or contamination, the device comprising a pair of bandages, each bandage comprising a sheet of metal or conducting polymer, wherein each bandage forms a single half-cell and comprises the same metal or conducting polymer, and wherein the bandages are configured for placement on a patient, such that when a first of the pair is placed directly over a wound and a second of the pair is placed on nearby skin over a wound-free area, the skin acts as a low impedance conductor and a connected circuit detects a voltage differential between each half-cell.

4. The device of any of the preceding claims, further comprising a detection circuit, which comprises an amplifier, a processor, and memory.

5. The device of claim 4, wherein the detection circuit is separably connected to the wrapping via a connector.

6. The device of claim 4, wherein the detection circuit is permanently disposed on or in the wrapping.

7. The device of claim 5 or 6, further comprising a wireless transmitter.

8. A system comprising:
the device of any of the preceding claims; and
a receiving circuit configured to further process and/or analyze voltage signals recorded by the wrapping.

9. The system of claim 8, wherein the receiving circuit comprises a wireless receiver, a processor, a memory, and an output display.

10. A method of identifying active microbial growth in or on a specimen, comprising recording voltage at the specimen over time and identifying a change in voltage, wherein an increase in voltage is indicative of: a decrease in pH, an increase in active microbial growth, and an incipient microbial infection or contamination.

11. The method of claim 10, further comprising placing a wrapping on the specimen, wherein the wrapping comprises an active region and a reference region, each region having a positive node and a negative node, and wherein placing the wrapping on the specimen comprises positioning the active region on the specimen and the reference region at a location near the specimen.

12. The method of claim 11, further comprising subtracting a voltage recorded at the reference site from a voltage signal recorded at the active site to minimize the effect of environmental variables on the voltage signal and thereby improve a signal-to-noise ratio.

13. The method of claim 10, further comprising placing an active bandage on the specimen, the specimen being a wound, and placing an identical reference bandage on a skin surface separate from but near the wound, wherein each bandage comprises a sheet of conducting material.

14. The method of claim 13, wherein recording voltage comprises recording the voltage difference between the active and reference bandages.

15. The method of any of claims 10-14, further comprising generating an output signaling a decrease in pH, an increase in active microbial growth, and/or an incipient microbial infection or contamination.

16. The method of any of claims 10-15, further comprising converting an analog voltage signal to a digital signal, and sending the digital signal to a computer for signal processing and analysis.

17. The method of claim 16, wherein sending the digital signal to a computer comprises transmitting the digital signal to a remote computer via a wireless transmitter.
